(19) United States
(12) Patent Application Publication
(10) Pub. No.: US 2002/0193781 A1
(43) Pub. Date: Dec. 19, 2002

(54) Devices for interstitial delivery of thermal energy into tissue and methods of use thereof

(76) Inventor: Marvin P. Loeb, Huntington Beach, CA (US)

Correspondence Address: OLSON & HIERL, LTD., 36th Floor, 20 North Wacker Drive, Chicago, IL, 60606, US

(21) Appl. No.: 09/881,190

(22) Filed: Jun. 14, 2001

(51) Int. Cl. A61B 18/04; A61B 18/14; A61B 18/20

(52) U.S. Cl. 606/15; 606/33; 606/41

(57) ABSTRACT

A device is provided for interstitial delivery of thermal energy and/or a biologically compatible bulking material into tissues in a confined space. The device include means for delivering laser, radio-frequency, electrical, microwave, ultrasound or other thermal energy, as well as a port and channel for concomitant or subsequent injection of a bulking agent. The method of use of the device in the treatment of female stress incontinence (FSI), gastro-esophageal reflex disease (GERD), benign prostate hyperplasia (BPH) and other conditions is described.

### FIELD OF THE INVENTION

The invention relates to catheter devices. More particularly, the invention relates to catheter devices capable of interstitially delivering thermal energy to selected body tissues.

### BACKGROUND OF THE INVENTION

It is common to apply localized heating to tissue within patient's bodies to cauterize lesions and stop bleeding. Localized heating is also used to coagulate or vaporize tissues in a variety of medical procedures, for example, in the treatment of bleeding ulcers.

RF energy is frequently utilized for thermally coagulating tissues to stop bleeding. Other examples of the use of radio frequently (RF) heating devices can be found in cauterization of the endometrium of the uterus to treat excessive bleeding and the lobes of the prostate to treat benign prostate hyperplasia (BPH).

Optical fibers have been used to introduce laser energy into tissue to thermally vaporize, ablate, or coagulate tissue. Laser catheter devices have been utilized for the treatment of a number of disease conditions, such as destruction of plaque deposits in blood vessels, removal of neoplastic pulmonary tissue and cauterization of the endometrium. U.S. Pat. No. 5,649, 924 to Everett et al. describes a laser catheter for application of localized heat to body tissues. The device is useful for irradiation of the inner surface tissues of a body lumen or cavity, such as coagulation of the lobes of the prostate to treat BPH. An expandable laser catheter useful for removal of obstructions within a blood vessel is described in U.S. Pat. No. 5,466,234 to Loeb et al.

In order to interstitially deliver laser or RF energy into tissue, an optical fiber or RF electrode is inserted into the tissue by passing it through the channel of an articulating catheter or endoscope. Articulating catheters typically require a bend radius of 2 cm or more to reach an angle of 90 degrees from the axis of the catheter, and mechanical endoscope bridges are generally able to deflect optical fibers or RF electrodes only about 30 degrees from the axis of the endoscope. Such devices cannot be used in confined spaces without prying the tissues apart, which may be undesirable or impractical.

It would be desirable to be able to insert a device into tissue and interstitially heat, coagulate, or vaporize the tissue without thermally damaging the sensitive mucosa or endothelial lining of the tissue through which the device is inserted. While such effects can be accomplished with conventional devices in areas that are easily accessible, when it is desired to insert a device at a sharp angle into the tissue surrounding a lumen or cavity such as a duct, hollow organ, or other confined spaces or cavities, conventional devices may not be able to do so. Such confined spaces and uses include, for example, the lobes of the prostate to treat BPH, the female urethra beneath the bladder to treat female stress incontinence (FSI), the esophagus in the area of the sphincter to treat gastro-esophagus reflux diseases (GERD), the vesico-uretal junction to treat vesco-uretal reflux (VUR). Other applications include reaching difficult to access tumors.

It is an object of this invention to be able to interstitially treat tissues in a confined space with simple, reliable devices with little or no moving parts, which do not require computerized controls, and which, if needed, can be used through conventional viewing endoscopes and similar devices.

### SUMMARY OF THE INVENTION

A catheter device is provided that is suitable for insertion into a target tissue and for interstitially heating, coagulating or vaporizing the target tissue without thermally damaging the sensitive mucosa or endothelial lining of the tissue through which the device is inserted. The catheter device includes a hollow introducer sheath open at both ends, a permanently curved, but flexible metal cannula receivable within the introducer sheath; and a flexible electromagnetic energy conduit within the flexible metal cannula, said conduit having a proximal end adapted for connection to an energy source and a distal end adapted for delivering thermal energy to a predetermined tissue site.

As described herein, the term “introducer sheath” refers to any generally tubular device, suitable for medical use, such as a catheter, a channel of an endoscope or like device.

The flexible metal cannula made of a superelastic shape memory alloy, such as nitinol, manufactured by Memry, Corporation of Bethel, Conn., USA, and has a beveled distal end, for example, like that of a syringe needle, to facilitate tissue penetration. At its distal end portion, the flexible metal cannula has a radius of curvature of less than 2 cm to achieve a 90 degree angle from the axis of the introducer sheath when unconstrained by the introducer sheath. Superelasticity imparts a rubber-like mechanical flexibility to the cannula, which allows it to be readily deformed from its prefabricated, curved memory-shape by confinement in the relatively more rigid introducer sheath. That is, the radius of curvature of the distal end portion of the flexible metal cannula is decreased when the end portion is disposed and constrained within the introducer sheath. Devices made from superelastic metal alloys can pass through sharp curves and bends without themselves being permanently bent or kinked. This property is referred to as shape memory.

When confined in the channel of the introducer sheath, the flexible metal cannula can be straightened without exceeding the elastic limit thereof so as to conform to the bends and turns of the channel as the introducer sheath is flexed. However, when the distal end of the flexible metal cannula exits the channel of the introducer sheath, it immediately reverts to its prefabricated curved memory-shape. No mechanical means or computerized control system is needed to effect this change of shape.

The electromagnetic energy conduit is preferably a fiber optic. Alternatively, the energy conduit may be a wire assembly, such as a pair of electrical leads, operably connected to an energy emitting device at its distal end. Preferred energy emitting devices include RF electrodes, resistive heating loops, ultrasound generators, microwave energy generators, and the like.

In a method aspect of the present invention, the present catheter device is inserted into a body lumen, hollow organ or body cavity of a patient, such as the esophagus, urethra, or any other such tissue accessible via catheter, endoscope or like medical devices. The catheter is manipulated through the lumen or cavity to a predetermined position within the patient's body. The distal end portion of the flexible metal cannula is then moved forward through the introducer sheath so that the end portion of the flexible metal cannula exits the introducer sheath. The flexible metal cannula end portion then immediately returns to its prefabricated, curved memory shape. When the curved end portion of the cannula exits the distal end of the introducer sheath it encounters the epithelium or lining of the lumen or cavity and penetrates therethrough into the underlying tissue. The degree of penetration is controlled by how far the cannula end portion is advanced from the introducer tube. The distal end of the cannula is beveled in the form of a syringe needle to facilitate insertion into the tissue.

The flexible electromagnetic energy conduit can likewise be advanced through the cannula, after the flexible metal cannula has penetrated the tissue, such that the distal end of the conduit exits the cannula and penetrates further into the tissue.

The flexible energy conduit is preferably a fiber optic adapted for delivery of infrared (IR), ultraviolet (UV) or visible coherent radiation or light. The flexible energy conduit may alternatively be a wire assembly, or pair of leads, the distal end of which is connected to an RF electrode, a resistive heating loop, or an ultrasound or microwave energy generator. Electromagnetic energy is next applied to the tissue through the energy conduit in a manner that heats the tissue in contact with or in front of the distal end of the conduit. When the energy conduit is a fiber optic, the electromagnetic energy is supplied by IR, UV or visible coherent radiation. When the energy conduit is a wire assembly operably connected to an energy emitting device at its distal end, thermal energy is generated in the tissue by the supply of electric current to the energy generating device.

Irradiation of the tissue is affected at an energy level, and for a period of time, which is therapeutically effective for the disease condition being treated. The applied energy may be utilized to cauterize, coagulate, vaporize or ablate tissue without damage to overlying sensitive tissues lining the body lumen, hollow organ or cavity. When application of localized thermal energy is complete, the cannula and energy conduit portions of the present catheter device may be easily withdrawn from the tissue and retracted into the introducer sheath so that the whole device may be safely withdrawn from the body lumen, hollow organ or cavity, or surgically created passageway.

The method of the present invention may be used for the treatment of a number of disease conditions including prostatic enlargement, called benign prostatic hyperplasia, female stress incontinence, gastro-esophageal reflux disease, vesico-uretal reflex, tumors, and other conditions wherein treatment can effected by localized, interstitial heating and the targeted tissue is accessible from a body lumen, hollow organ or cavity or surgically created passageway.

Numerous other advantages and features of the present invention will be readily apparent to those of skill in the art from the drawings, the detailed description of the preferred embodiments and the appended claims.

### BRIEF DESCRIPTION OF THE DRAWINGS

In the drawings:

FIG. 1 is a partial, cross-sectional view of a catheter device embodying the present invention.

FIG. 2 is a partial, cross-sectional view of the catheter device of FIG. 1, shown positioned within the urethra of a patient, with the flexible metal cannula end portion constrained within the introducer sheath.

FIG. 3 is a partial, cross-sectional view of the catheter device of FIG. 1, shown positioned within the urethra of a patient, with the flexible metal cannula end portion extended out of the introducer sheath into the tissue surrounding the urethra.

FIG. 4 is a partial, cross-sectional view of a catheter device of the invention, wherein the introducer sheath is an endoscope, and the device is positioned in the esophagus of a patient in the area of the sphincter, with the flexible metal cannula extending from the distal opening of a channel of the endoscope into the target tissue, with the flexible energy conduit further extended out of the flexible metal cannula into the target tissue.

FIG. 5 is a partial cross-sectional view of the another embodiment of the device of FIG. 1, having a port for infusion of fluid or drawing a vacuum through the flexible metal cannula and plastic sleeve disposed over the flexible metal cannula, shown without the introducer sheath.

FIG. 6 is a cross-sectional view of the device of FIG. 5, wherein the introducer sheath is a channel of an endoscope, shown with the distal end portion of the flexible metal cannula disposed within the endoscope channel.

FIG. 7 is a cross-sectional view of the device of FIG. 6, shown with the distal end portion of the flexible metal cannula extended out of the distal opening of the channel of the endoscope into the surrounding tissue.

### DETAILED DESCRIPTION OF PREFERRED EMBODIMENTS

While this invention can be embodied in many different forms, there are preferred embodiments shown in the drawings and described in detail. The present disclosure is an exemplification of the principles of the invention and is not intended to limit the invention to the embodiments illustrated.

An apparatus aspect of the present invention is a medical catheter device for delivering localized thermal energy to a tissue in a patient's body. In use, the catheter device is suitably positioned within a patient's body by insertion through a body lumen or cavity and advanced to a predetermined site within the body. A flexible metal cannula portion of the catheter device is extended into the predetermined site within the tissue surrounding the lumen or cavity. Localized energy is then applied to the tissue by an electromagnetic energy conduit within the cannula portion. The applied energy may be utilized to cauterize, coagulate, vaporize, ablate or cut the tissue without damage to overlying sensitive tissues lining the lumen or cavity. After electromagnetic energy is applied to the tissue, the metal cannula and energy conduit may be withdrawn into the catheter device, which may then be safely withdrawn from the lumen or cavity.

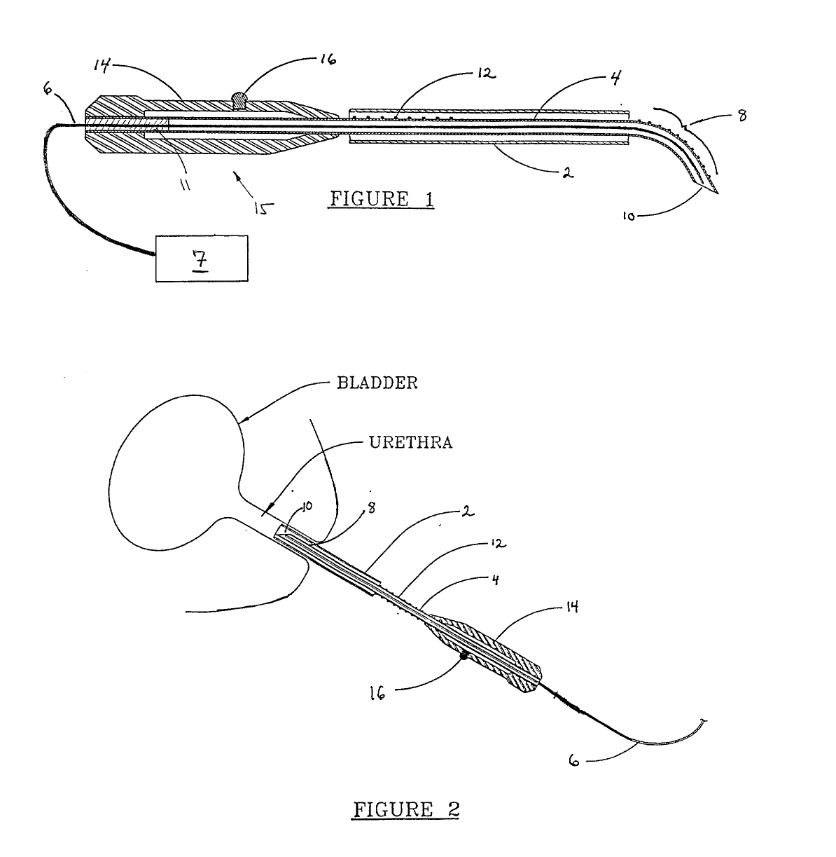

FIG. 1 illustrates one embodiment of the catheter device of the present invention. Introducer sheath 2 comprises a flexible or rigid tube such as a plastic catheter, or a channel of a rigid endoscope. A flexible metal cannula 4 is disposed within introducer sheath 2. Preferably, flexible metal cannula 4 is made of a superelastic shape memory alloy which is a substantially equimolar alloy of nickel and titanium commonly referred to as a nitinol, available from Memory Corporation of Bethel, Conn., USA. Distal end 10 of metal cannula 4 can be beveled in the manner of a syringe needle to facilitate insertion into body tissue. Metal cannula 4 is slidably moveable within introducer sheath 2. Distal end portion 8 of metal cannula 4 is permanently curved by earlier having been heated above its transition temperature and is shown in its position extended from introducer sheath 2. Preferably, the unencumbered distal end portion 8 of metal cannula 4 has a radius of curvature of less than about 2 cm when bent at a 90 degree angle from the axis of the cannula.

Flexible electromagnetic energy conduit 6 is fixably attached within flexible metal cannula 4 by adhesive plug 11. In this particular embodiment, flexible energy conduit 6 is a fiber optic adapted for delivery of infrared, eximer or visible laser light, optically coupled to laser energy source 7. Alternatively, flexible electromagnetic energy conduit 6 may be a wire assembly electrically connected at its proximal end to a source of RF or electrical energy and at its distal end to an RF electrode, resistive electrical heating loop or an ultrasonic or microwave generator capable of generating thermal energy in a tissue upon application of electrical energy thereto.

Optional handpiece 14 is fixably attached to the proximal end portion 15 of flexible metal cannula 4 by an adhesive, solder, weld, compression fitting or other suitable means therefor. Handpiece 14 may be composed of any material suitable for medical applications, but is preferably made of a plastic, such as Teflon® fluorinated hydrocarbon, a polycarbonate, a polystyrene or the like. Optional indicator 16 on handpiece 14 may be a button, knob, ridge or other marking disposed in a fixed position relative to the direction of curvature of the distal end portion 8 of the flexible metal cannula 4. Indicator 16 allows the operator of the device to know in which direction the distal end portion 8 of cannula 4 will enter the tissue when it is in its extended position. Optional markings 12, on the portion of cannula 4 extending distally from handpiece 14, allow the operator of the device to gauge, knowing the length of introducer sheath 2, the distance that the distal end portion 8 of cannula 4 has moved out of the introducer sheath 2.

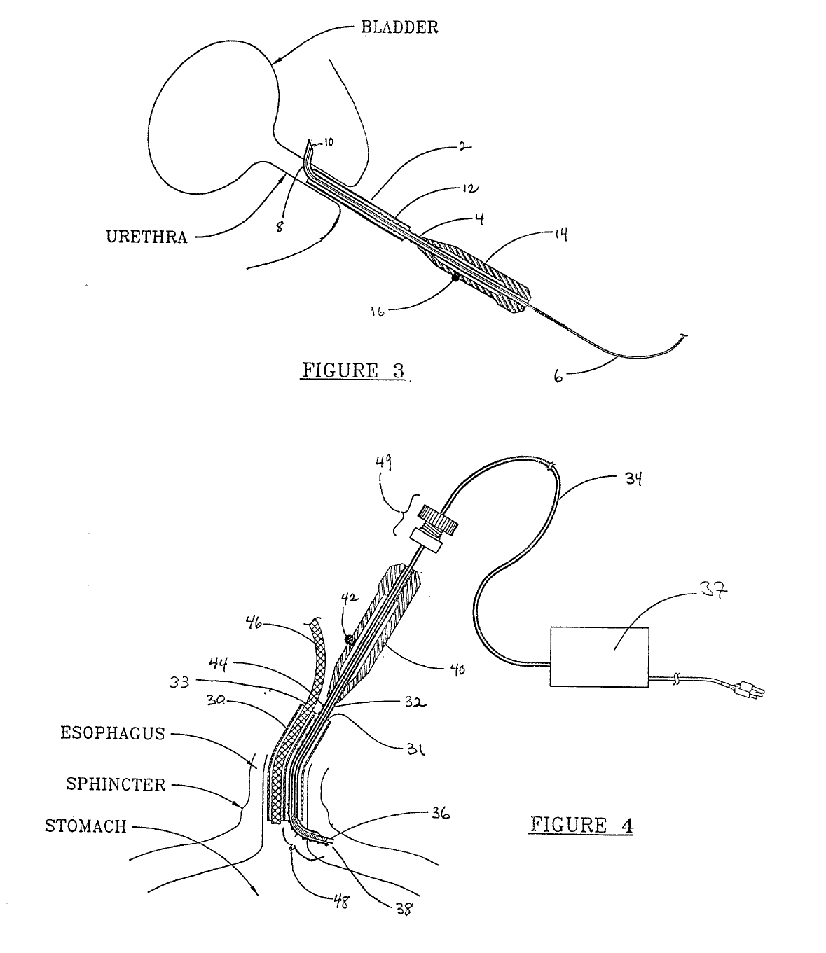

FIGS. 2 and 3 illustrate the catheter device of FIG. 1 as it would be deployed in a typical medical procedure, as for example, in the female urethra, beneath the bladder, to treat FSI. In FIG. 2, proximal end portion 8 of cannula 4 is shown in its confined, straight position within introducer sheath 2. With the end portion 8 of cannula 4 in its confined position within introducer sheath 2, whose wall strength exceeds the force exerted by the pre-formed curved shape of proximal end portion 8 of cannula 4, introducer sheath 2 may be inserted into a body lumen or cavity.

In FIG. 3, cannula 4 has been advanced beyond the distal end of introducer sheath 2, and end portion 8 of cannula 4 has resumed its preformed curved shape and penetrated the urethra into the surrounding tissue.

In the position illustrated in FIG. 3, the tissue may be heated by supplying energy through energy conduit 6 thereto. When irradiation of the tissue is complete, energy conduit 6 and cannula end portion 8 are retracted back into introducer sheath 2 to the position illustrated in FIG. 2, after which introducer sheath 2, containing cannula 4 and energy conduit 6, may be withdrawn from the body lumen or cavity or moved to another position therein for further thermal treatments. In FIGS. 2 and 3, indicator 16 is fixedly positioned on the surface of handpiece 14 opposite the direction of curvature of end portion 8 of cannula 4, thus indicating to the operator the direction that cannula 4 will take when advanced out of the introducer sheath 2 into the tissue surrounding the body cavity or lumen. Indicator 16 may be positioned on the same side of handpiece 14 as the direction of curvature of end portion 8, or in any other fixed position as may be convenient to the operator.

Indicator 16 is optional, in that the direction of entry of the end portion 8 of cannula 4 into tissue may be determined by external means such as endoscopic viewing or ultrasonic imaging. Likewise, it is only necessary that the operator know the relationship between the markings 12 and the length of introducer sheath 2 to determine the distance cannula 4 has penetrated into the tissue.

FIG. 4 illustrates an embodiment of the present invention in which the introducer sheath 30 is a channel of an endoscope and wherein the energy conduit 34 is a pair of insulated electrical wires or leads adapted at their proximal end for connection to a source of RF energy and the distal ends of which are operably connected to bipolar RF electrodes 36 and 38 capable of generating localized heating in the tissue into which electrodes 36 and 38 are inserted. In FIG. 4, the device is illustrated positioned for use in the esophagus of a patient, for example, to treat gastro-esophageal reflux disease or GERD.

The device in FIG. 4 is positioned for use within an Amendoscope 30, having a channel 31 functioning as an introducer sheath, and channel 33 for viewing and other channels (not shown) for illumination of the tissue and other purposes. Flexible metal cannula 32, confined within channel 31, is made of a superelastic shape memory alloy whose distal end portion has earlier been fixed in a curved shape by heating it above its transition temperature. Flexible energy conduit 34, comprising a pair of insulated wire leads, the proximal ends of which are connected to RF energy source 37, and distal ends of which terminate in electrodes 36 and 38 to form a bipolar RF electrode, is slidably disposed within cannula 32.

Handpiece 40 is fixably attached to cannula 32 as described in the device of FIG. 1. Indicator 42 is positioned on the side of handpiece 40 opposite the direction of curvature of end portion 48 of cannula 32 so as to indicate to the operator the direction that the distal end of cannula 32 will enter the surrounding tissue. Optional markers 44, on the proximal end portion of cannula 32, just distal to handpiece 40, are visible to the operator of the device and serve to allow the operator to determine the distance that end portion 48 of cannula 32 has been advanced out of channel 31 of endoscope 30.

An optical viewing means, such as a fiber optic 46, may optionally be inserted in channel 33 of endoscope 30, so that the area of the body lumen or cavity near the distal end of endoscope 30 may be viewed. Optionally, a stop assembly 49 may be fixably attached to the flexible energy conduit proximal to handpiece 40 to limit the extent to which flexible energy conduit 34 may be extended out of the distal end of channel 31. Stop assembly 49, as known in the art, may consist of a compressible plastic fitting with threads of increasing outside diameter and a threaded nut which, when screwed onto said plastic fitting, causes it to compress upon conduit 34.

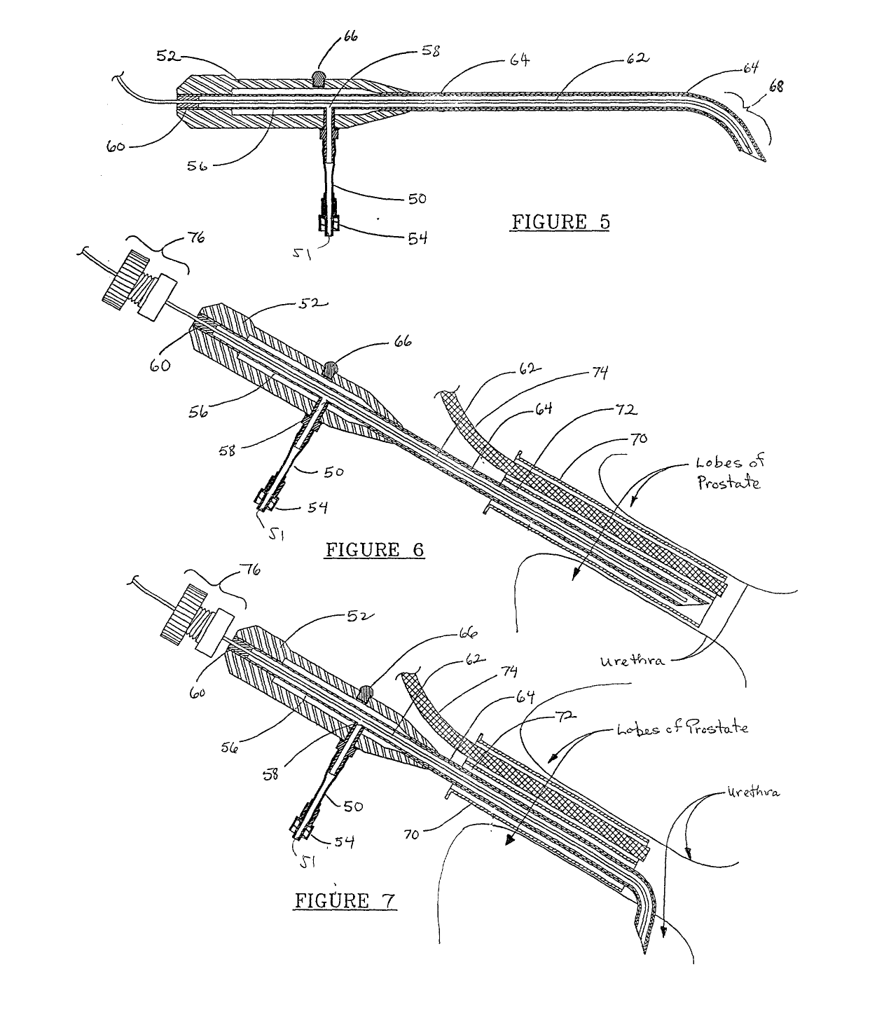

Another embodiment of the catheter device of the present invention is shown in FIGS. 5, 6 and 7. FIG. 5 shows a partial cross-sectional view of a laser catheter device, without the introducer sheath, similar to that depicted in FIG. 1, but having additional features that allow for the pumping of fluids into and out of the tissue that is being thermally treated. The device illustrated in FIG. 5 is provided with hollow stem 50 which defines port 51, for fluid transport or vacuum. Luer lock assembly 54 is provided on stem 50 around port 51. A channel 58 is provided in the interior of flexible metal cannula 56, such that port 51 is in fluid communication with channel 58. The proximal end of channel 58 in cannula 56 contains a gasket 60, that creates a fluid tight seal with the extension of flexible energy conduit 62. As a result, flexible energy conduit 62 can be slidably movable through gasket 60, without breaking the fluid tight seal.

Optionally, a polymeric coating or sleeve 64 may be attached to the outer surface of flexible metal cannula 56. Coating or sleeve 64 facilitates penetration of cannula 56 into tissue, prevents tissue from adhering to cannula 56 and thermally insulates cannula 56, preventing undesired thermal damage to tissue through which cannula 56 extends, which may otherwise be caused by thermal conduction through metal cannula 56. Coating or sleeve 64 also protects flexible metal cannula 56 from damage or binding when it is inserted into or withdrawn from an introducer sheath such as a catheter or endoscope. Preferably, polymeric coating or sleeve 64 comprises a fluorocarbon polymer such as poly(tetrafluoro)ethylene (e.g. TEFLON®), available from DuPont de Nemours of Wilmington, Del. The flexible energy conduit 62 is preferably an optical fiber capable of delivering laser light energy to the tissue into which cannula 56 is inserted. Alternatively, flexible energy conduit 62 may comprise one or more wire leads connected to a thermal energy generator as described in the device of FIG. 4 hereinabove. The distal end portion 68 of flexible metal cannula 56 has been formed into a curved shape, but is sufficiently flexible to divert from its curved shape when constrained inside a relatively more rigid structure, such as a catheter or endoscope channel. Preferably the end portion 68 of cannula 56 has a radius of curvature of less than 2 cm when unconstrained.

FIGS. 6 and 7 show the device of FIG. 5 as it would be used in treatment of benign prostatic hyperplasia or BPH by interstitially vaporizing a portion of the excess tissue in the lobes of the prostate. As seen in FIG. 6, the device of FIG. 5 is inserted into endoscope 70 through channel 72. FIG. 6 depicts the flexible metal cannula 56 in its constrained position, with the curved end portion 68 of cannula 56 having straightened due to constraint within channel 72. Endoscope 70 is disposed in the urethra of a male patient with the distal end of endoscope 70 terminating just below or within the portion of the urethra transecting the prostate.

As shown in FIG. 7, once endoscope 70 has been properly positioned within the prostate, flexible metal cannula 56, is advanced through channel 72, such that end portion 68 of cannula 56 exits the distal opening of channel 72. As it exits channel 72, end portion 68 of cannula 56 resumes its curved shape, penetrates the urethra and enters the underlying prostate tissue. Flexible conduit 62 may be extended forward into the tissue, beyond the distal opening of cannula 56. Optionally, stop assembly 76, as described in FIG. 4, may be removeably attached to flexible energy conduit 62 proximal to handpiece 52 to limit the extent to which flexible energy conduit 62 may be extended out of the distal end of channel 72. Optionally, as shown, coating or sleeve 64 may be affixed to the exterior of metal cannula 56 to facilitate its penetration into tissue, as described in FIG. 5.

With the distal end of optical fiber 62 about 0.5 cm inside the prostate tissue, about 50 watts of Holmium laser energy, for example 2.5 joules per pulse at a repetition rate of 20 pulses per second, may be emitted from optical fiber 62 for 15 to 30 seconds, creating a spherical vaporization zone approximately 0.5 to 1.0 cm in diameter within the tissue, without damaging the urethra or the 0.5 cm of tissue immediately underlying the urethra, which is essential to the urethra's blood supply and survival. Maintaining the integrity of the urethra avoids the sloughing of tissue, irritative symptoms and dysuria that occurs if laser energy is emitted interstitially in a lobe of the prostate within 0.5 cm of the urethra, or when laser energy is transmitted through the urethra to coagulate the underlying tissue of the lobe of the prostate.

Alternatively, with the device similarly positioned, 60 watts of Holmium laser energy, for example 3 joules per pulse at a repetition rate of 20 pulses per second, can be emitted from optical fiber 62 for 15 to 30 seconds to create a spherical vaporization zone of approximately 0.75 to 1.5 cm in diameter.

Depending on the size of the prostate, about four to twenty or more insertion points may be employed to treat BPH, applied at, for example, 2, 4, 8 and 10 o'clock, in a series of circumferential lasings or in any other desired pattern, as more fully described in co-owned U.S. Pat. No. 5,649,924, which is fully incorporated herein by reference.

If a lesser amount of laser energy is applied, the period of energy emission must be longer to create a similarly sized vaporization zone. However, with a longer emission period, greater time for conduction of heat and a larger coagulation zone will result, which could damage the tissue immediately underlying the prostate and jeopardize its survival, resulting in the aforementioned dyusia and other adverse effects. Excessive coagulation also creates significant edema of the tissue, which causes swelling of the prostate lobes and can take days to subside, requiring the use of a drainage catheter for a number of days to enable the patient to urinate.

While, high intensity incoherent light or Argon, KTP, Nd:YAG and similar visible and near infrared lasers may be used to treat BPH in the manner described herein, such lasers are not as effective as the Holmium: YAG laser or an excimer laser in vaporizing tissue. Argon, KTP, diode, Nd:YAG and similar lasers also create a much larger coagulation zone, increasing the amount of edema in the lobes of the prostate and the adverse effects described above. Thus, a Holmium laser is preferred for the treatment of BPH.

However, Argon, KTP, diode, Nd:YAG and similar visible and near-infrared lasers or a source of high intensity incoherent light, operably coupled to the devices of the present invention, may be used to interstitially heat, shrink and coagulate the tissue underlying the female urethra to treat FSI, the esophagus in the area of the sphincter to treat GERD or the vesico-uretal function to treat VUR, utilizing 5 to 25 watts of power for 2 to 30 seconds at each insertion point. About two to eight or more insertion points may be required to treat FSI, and about four to twenty or more insertion points may be employed to treat GERD or VUR. Generally, these may be applied, for example, at 3, 6, 9 and 12 o'clock, at 2, 4, 8 and 10 o'clock, or in any other desired pattern.

The catheter devices of the present invention may also be utilized for the treatment of difficult to access tumors. The devices may be inserted through a naturally occurring or surgically created passageway and placed alongside or within the tumor. The flexible metal cannula and flexible energy conduit can be advanced into the tissue and energy emitted to heat, coagulate or vaporize the tumor, in a manner similar to that described hereinabove for the treatment of BHP.

While conventional optical fibers can be used with Argon, KTP, Nd:YAG and similar lasers, it is necessary to use optical fibers with a low-hydroxyl (low-OH) content if Holmium laser energy is to be utilized with the devices of the present invention. Optical fibers with a high hydroxyl (high-OH) are generally required if excimer laser energy is to be used with the present invention. Preferably, the core diameter of optical fibers useful as flexible energy conduits in the devices of the present invention are in the range of about 100 to 600 microns, preferably 100 to 400 microns for the treatment of FSI, about 200 to 600 microns to treat GERD or VUR, and 300 to 600 microns to treat BPH.

In addition to laser energy, the flexible energy conduit may be adapted to deliver RF energy, electrical energy, microwave or ultrasound energy to treat conditions such as FSI, GERD or VUR. To treat these conditions, about 3 to about 50 watts, preferably about 5 to about 60 watts of power, is emitted for about 2 to 30 seconds at each insertion point, with the insertion points disposed as described above.

Preferably, flexible metal cannulas 4, 32 and 56 are composed of a shape memory alloy such as nitinol, which is a substantially 1:1 alloy of nickel and titanium. Nitinol generally has an atomic ratio of nickel to titanium in the range of about 49:51 to about 51:49. Nitinol alloys may also comprise about 0.1 to about 5% by weight of other elements such as iron, chromium and copper.

A syringe or pump may be attached to port 50 via luer lock assembly 54, and sterile water, saline or other biocompatible liquid may be infused through the channel 56 of the embodiment described in FIGS. 5, 6 and 7 hereinabove, to cool the distal end of the device during operation of the laser. Saline is the preferred fluid if RF energy is to be utilized. Alternatively, in the treatment of BPH, or other treatments wherein tissue vaporization is desired, a vacuum may be applied to port 50 via luer lock assembly 54 to remove hot gasses from the vaporization of tissue. Removal of hot gases can reduce the coagulation zone, edema and other adverse effects.

In the treatment of FSI, GERD or VUR, a biologically compatible bulking material, including bovine collagen, such as Contigen® distributed by C. R. Bard, Inc. of Murray Hill, N.J., microspheres or a biologically inert material, may optionally be injected into the tissue through port 50 of the device depicted in FIGS. 5, 6 and 7. Injection of a bulking material can be useful the treatment of conditions such as FSI, GERD and VUR, by expanding the tissue interstitially and cause tightening of the tissue underlying the female urethra, the esophageal sphincter or the vesico-uretal junction.

If low level laser, RF, electrical, microwave, ultrasound or other thermal energy is emitted during or after the injection of collagen, the temperature of the collagen may be raised to about 50 to about 75° C., preferably about 55 to about 65° C., to cause cross linking of the collagen, which reduces its propensity to migrate away from the injection site.

As can be seen from the drawings and the above descriptions, a device for delivering thermal energy and/or bulking materials interstitially in a confined space is provided, which avoids the need to pry apart the tissues or utilize a mechanical device.

Numerous variations and modifications of the embodiments described above may be effected without departing from the spirit and scope of the novel features of the invention. It is to be understood that no limitation with respect to the specific apparatus illustrated herein is intended or should be inferred. It is, of course, intended to cover by the appended claims all such modifications as fall within the scope of the claims.

I claim:

1. A catheter device adapted for delivering thermal energy to a body tissue, having a proximal end for connection to an energy source, a tubular body portion, and a distal end adapted for penetration into body tissue, which catheter device comprises:
(a) a permanently curved flexible metal cannula made of a superelastic shape memory alloy, and having a beveled distal end capable of penetrating body tissue; and
(b) a flexible energy conduit within the flexible metal cannula and adapted for delivering energy to a predetermined tissue site; said metal cannula being sufficiently flexible to be straightened when confined within a relatively more rigid passageway without exceeding the elastic limit thereof.

2. The catheter device of claim 1, wherein the flexible energy conduit is a fiber optic, having a proximal end adapted for connection to a radiant energy source.

3. The catheter device of claim 2, wherein the fiber optic comprises at least one optical fiber having a core diameter in the range of about 100 microns to about 600 microns.

4. The catheter device of claim 1, wherein the energy conduit comprises at least one wire capable of transmitting radiofrequency energy from a source of radiofrequency energy to at least one electrode capable of delivering radiofrequency energy to a body tissue.

5. The catheter device of claim 1, wherein the energy conduit comprises a pair of leads having proximal ends adapted for connection to an electrical power source and distal ends operably connected to an ultrasonic generator.

6. The catheter device of claim 5, wherein the ultrasonic generator is a piezoelectric generator.

7. The catheter device of claim 5, wherein the ultrasonic generator is a magnetostrictive generator.

8. The catheter device of claim 1, wherein the energy conduit comprises a pair of leads having proximal ends adapted for connection to an electrical power source and a distal end portion operably connected to a resistive heating loop.

9. The catheter device of claim 1, wherein the energy conduit comprises a pair of leads having proximal ends adapted for connection to an electrical power source and distal ends operably connected to a microwave energy source.

10. The catheter device of claim 1, wherein the flexible metal cannula comprises a superelastic shape memory alloy of nickel and titanium, having a nickel:titanium atomic ratio in the range of about 49:51 to about 51 to 49.

11. The catheter device of claim 10, wherein the nickel-titanium alloy further comprises about 0.1% to about 5% by weight of a metal selected from the group consisting of iron, chromium and copper.

12. The catheter device of claim 1, wherein the metal cannula further comprises a hollow, substantially cylindrical handpiece coaxially disposed over the proximal end portion of the metal cannula and affixed thereto.

13. The catheter device of claim 12, wherein the handpiece further comprises a marker which is positioned in a fixed relationship to the direction of the curvature of the distal end portion of the metal cannula.

14. The catheter device of claim 12, wherein the handpiece further defines a tubular port having an exterior end adapted for connection to a source of fluid, and said tubular port being in fluid flow communication with the metal cannula providing a channel from the exterior of the handpiece to the interior of the flexible metal cannula, such that a fluid may be passed through said port.

15. The catheter device of claim 14, wherein said handpiece is adapted for connection of the port to a vacuum source.

16. The catheter device of claim 1, wherein the flexible metal cannula has a polymeric sleeve on its exterior surface.

17. The catheter device of claim 16, wherein the polymeric sleeve is made of a fluorocarbon polymer.

18. The catheter device of claim 1, wherein the flexible metal cannula has a polymeric coating on its exterior surface.

19. The catheter device of claim 18, wherein the polymeric coating is a fluorocarbon polymer coating.

20. A catheter device adapted for delivering energy to heat a body tissue, having a proximal end for connection to an energy source, a tubular body portion, and a distal end adapted for penetration into body tissues, which catheter device comprises;
(a) an introducer sheath which is a hollow tube open at both ends;
(b) a permanently curved flexible metal cannula, receivable within the introducer sheath, having a beveled distal end capable of penetrating body tissues when the distal end of the metal cannula extends beyond the distal end of the introducer sheath; and
(c) a flexible energy conduit within the flexible metal cannula adapted for delivering energy to a predetermined tissue site.

21. The catheter device of claim 20, wherein the flexible metal cannula is made of a superelastic shape memory alloy and at the distal end portion thereof having a radius of curvature of less than 2 cm, said flexible metal cannula being temporarily straightenable when confined within a relatively more rigid introducer sheath.

22. The catheter device of claim 20, wherein the flexible energy conduit is a fiber optic, capable of delivering light energy to a body tissue, and having a proximal end adapted for connection to a light source.

23. The catheter device of claim 22, wherein the fiber optic comprises at least one optical fiber having a core diameter in the range of about 100 microns to about 600 microns.

24. The catheter device of claim 20, wherein the energy conduit comprises at least one wire whose proximal end is adapted for connection to a source of radiofrequency energy and whose distal end comprises at least one electrode capable of delivering radiofrequency energy to a body tissue.

25. The catheter device of claim 20, wherein the energy conduit comprises a pair of leads having proximal ends adapted for connection to an electrical power source and distal ends operably connected to an ultrasonic energy generator.

26. The catheter device of claim 25, wherein the ultrasonic generator is a piezoelectric generator or a magnetostrictive generator.

27. The catheter device of claim 20, wherein the energy conduit comprises a pair of leads having proximal ends adapted for connection to an electrical power source and a distal end portion operably connected to a resistive wire heating loop.

28. The catheter device of claim 20, wherein the energy conduct comprises a pair of leads having proximal ends adapted for connection to a source of electrical energy and distal ends operably connected to a microwave energy generator.

29. The catheter device of claim 20, wherein the metal cannula further comprises a hollow, substantially cylindrical handpiece coaxially disposed over the proximal end portion of the metal cannula and affixed thereto.

30. The catheter device of claim 29, wherein the handpiece further defines a tubular port in fluid communication with the cannula and adapted for connection to a pump, such that a fluid may be passed through said port.
